(19) United States
(12) Patent Application Publication
(10) Pub. No.: US 2021/0358585 A1
(43) Pub. Date: Nov. 18, 2021

(54) METHOD AND SYSTEM FOR CANCER STAGE ANNOTATION WITHIN A MEDICAL TEXT

(71) Applicant: KONINKLIJKE PHILIPS N.V., EINDHOVEN (NL)

(72) Inventors: Qingxin Wu, Lexington, MA (US); Woei-Jye Yee, Boston, MA (US); Robbert Christiaan van Ommering, Cambridge, MA (US); Samuel Frank Pilato, Cambridge, MA (US)

(21) Appl. No.: 17/272,570

(22) PCT Filed: Aug. 27, 2019
(86) PCT No.: PCT/EP2019/072816

§ 371 Date: Mar. 1, 2021

Related U.S. Application Data
(60) Provisional application No. 62/723,645, filed on Aug. 28, 2018.

(51) Int. Cl. G16H 15/00; G16H 10/60; G16H 50/20

(52) U.S. Cl. CPC G16H 15/00; G16H 50/20; G16H 10/60

(57) ABSTRACT

A method (100) for generating a standardized cancer stage from a text-based source using an annotation system (400), comprising: (i) extracting (130), by a stage annotator, information from the text-based source relative to a stage of the patients cancer to generate cancer annotations; (ii) identifying (140), by a disease annotator, information from the text-based source indicative of a type of cancer; (iii) extracting (150), by a stage synonym annotator, information from the text-based source synonymous with a cancer to generate cancer annotations; (iv) converting (160), by a stage canonicalizer, the cancer annotations from the stage annotator and the stage synonym annotator to a standardized cancer stage; and (v) reporting (170) the standardized cancer stage, the report comprising the standardized cancer stage, the cancer annotations extracted sources from the text-based source, and/or the location of each of the cancer annotations within the text-based source.

### FIELD OF THE DISCLOSURE

The present disclosure is directed generally to methods and systems for characterizing and standardizing cancer stage information obtained from a document.

### BACKGROUND

Cancer stage is a critical attribute of cancer. For example, staging of cancer measures the size of a cancer and how far it has grown. Accordingly, staging information can assist medical professionals with the selection of optimal treatment. For example, when searching for eligible trials for a particular patient, the patient's cancer stage must match exactly with the cancer stage requirements of the trial as found in the eligibility criteria. However, there is no structured cancer stage information in clinical trials other than free text. Thus, detecting stage from entire clinical trial document and normalizing it are critical to clinical trial matching. However, manually extracting stage information from trials is time consuming, labor intensive, and error prone.

There are two main types of standardized staging systems for cancer. These are the TNM (Tumor, Node, and Metastasis) system, and numerical staging systems. A standardized staging system provides a number of benefits. First, medical professionals have a common language to describe cancer. Second, guidelines for treatment can be standardized between different medical treatment facilities. Additionally, treatment results can be accurately compared between research studies if a standardized staging system is used. In addition to these two main types of staging systems, there are several other ways to describe cancer stages which are not standardized. Some of these stage synonyms can be manually converted to one of the standardized staging systems, but there are no automated mechanisms for conversion. For example, a phrase such as ‘carcinoma in situ’ may equate to ‘stage 0’, while phrases such as ‘metastatic cancer’ and ‘advanced cancer’ are synonymous with ‘stage 4’.

Although staging information can be extremely beneficial, there is typically no structuralized stage information available because many kinds of clinical documents, including medical trial documentation, exist as free text and the stage information found in this free text is unstructured.

### SUMMARY OF THE DISCLOSURE

There is a continued need for methods and systems that automatically extract stage information from text-based documentation and convert the extracted stage information into a standardized format. Various embodiments and implementations herein are directed to a method and system configured to receive and process text-based sources, such as trial documentation or clinical documents, for text-based analysis. The system extracts information from the text-based source relative to a stage of the patient's cancer to generate one or more cancer annotations, comprising an identification of one or more locations within the text-based source with information indicative of a stage of cancer. The system identifies information within the text-based source indicative of a type of cancer, and extracts information from the text-based source synonymous with a cancer to generate one or more cancer annotations, if the synonymous information is determined, by a decision model, to closely relate to the type of cancer identified within the text-based source. The system converts the cancer annotations into a canonicalized, or standardized, cancer stage. The cancer stage is optionally reported out together with the cancer annotations extracted from the text-based source and/or the location of the one or more cancer annotations within the text-based source.

Generally, in one aspect, a method for generating a standardized cancer stage from a text-based source using an annotation system is provided. The method includes: (i) receiving a text-based source comprising information about a patient's medical state or condition; (ii) processing, by a processor, the text-based source for text-based analysis; (iii) extracting, by a stage annotator, information from the text-based source relative to a stage of the patient's cancer to generate one or more cancer annotations, comprising an identification of one or more locations within the text-based source comprising information indicative of a stage of cancer; (iv) identifying, by a disease annotator, information from the text-based source indicative of a type of cancer; (v) extracting, by a stage synonym annotator, information from the text-based source synonymous with a cancer to generate one or more cancer annotations, if the synonymous information is determined, by a decision model, to closely relate to the identified information indicative of a type of cancer; (vi) converting, by a stage canonicalizer, the one or more cancer annotations from the stage annotator and the stage synonym annotator to a standardized cancer stage; and (vii) reporting the standardized cancer stage, the report comprising the standardized cancer stage, the one or more cancer annotations extracted from the text-based source, and/or the location of each of the one or more cancer annotations within the text-based source.

According to an embodiment, the method further includes implementing an action based on the report. According to an embodiment, the action is implementation of a treatment plan by a healthcare professional. According to another embodiment, the action is identification of a suitable clinical trial for the patient based on the cancer stage extracted from the clinical trial.

According to an embodiment, the stage annotator comprises: (i) a TNM annotator configured to identify one or more locations within the text-based source comprising information indicative of a TNM classification of a tumor; and (ii) a number annotator configured to identify one or more locations within the text-based source comprising information indicative of numerical classification of a tumor.

According to an embodiment, the standardized cancer stage comprises a Roman numeral.

According to an embodiment, the method further includes testing the annotation system by: (i) generating, by a reviewer reviewing the text-based source, a standardized cancer stage; (ii) comparing the reviewer's standardized cancer stage to the standardized cancer stage generated by the annotation system; (iii) identifying, from the comparison, any differences between the reviewer's standardized cancer stage and the standardized cancer stage generated by the annotation system; and (iv) modifying one or more of the disease annotator, the stage annotator, the stage synonym annotator, and/or the stage canonicalizer if the reviewer's standardized cancer stage and the standardized cancer stage generated by the annotation system do not match.

According to an embodiment, the information from the text-based source synonymous with a cancer comprises information describing a physical state of a tumor.

In another aspect is a system configured to generate a standardized cancer stage from a text-based source. The system includes: a plurality of text-based sources; a processor configured to: (i) extract information from the text-based source relative to a stage of the patient's cancer to generate one or more cancer annotations, comprising an identification of one or more locations within the text-based source comprising information indicative of a stage of cancer; (ii) identify information from the text-based source indicative of a type of cancer; (iii) extract information from the text-based source synonymous with a cancer to generate one or more cancer annotations, if the synonymous information is determined to closely relate to the identified information indicative of a type of cancer; (iv) convert the one or more cancer annotations from the stage annotator and the stage synonym annotator to a standardized cancer stage; and (v) generate a report of the standardized cancer stage, comprising the standardized cancer stage, the one or more cancer annotations extracted from the text-based source, and/or the location of the one or more cancer annotations within the text-based source; and a user interface configured to communicate the report of the standardized cancer stage to a user.

According to an embodiment, the processor is configured to: (i) identify one or more locations within the text-based source comprising information indicative of a TNM classification of a tumor; and/or (ii) identify one or more locations within the text-based source comprising information indicative of numerical classification of a tumor.

According to an embodiment, the processor is configured to: (i) compare the standardized cancer stage to a standardized cancer stage generated by a human reviewer; (ii) identify any differences between the standardized cancer stage and the standardized cancer stage generated by the human reviewer; and (iii) modify the system if the standardized cancer stage and the standardized cancer stage generated by the human reviewer are not a match.

According to an embodiment, the plurality of text-based sources comprises clinical documents about one or more patients. According to another embodiment, the plurality of text-based sources comprises documentation about one or more clinical trials.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a flowchart of a method for standardizing cancer stage information, in accordance with an embodiment.

FIG. 2 is a flowchart of a method for standardizing cancer stage information, in accordance with an embodiment.

FIG. 3 is a flowchart of a method for standardizing cancer stage information, in accordance with an embodiment.

FIG. 4 is a schematic representation of a system for standardizing cancer stage information, in accordance with an embodiment.

### DETAILED DESCRIPTION OF EMBODIMENTS

The present disclosure describes various embodiments of a system and method for extracting stage information from text-based documentation and converting the extracted stage information into a standardized format. More generally, Applicant has recognized and appreciated that it would be beneficial to provide a system that standardizes cancer stage information extracted from text-based documentation. The system extracts information from the text-based source relative to a stage of the patient's cancer to generate one or more cancer annotations, comprising an identification of one or more locations within the text-based source with information indicative of a stage of cancer. The system identifies information within the text-based source indicative of a type of cancer, and extracts information from the text-based source synonymous with a cancer to generate one or more cancer annotations, if the synonymous information is determined, by a decision model, to closely relate to the type of cancer identified within the text-based source. The system converts the cancer annotations into a canonicalized, or standardized, cancer stage. The cancer stage is optionally reported out together with the cancer annotations extracted from the text-based source and/or the location of the one or more cancer annotations within the text-based source.

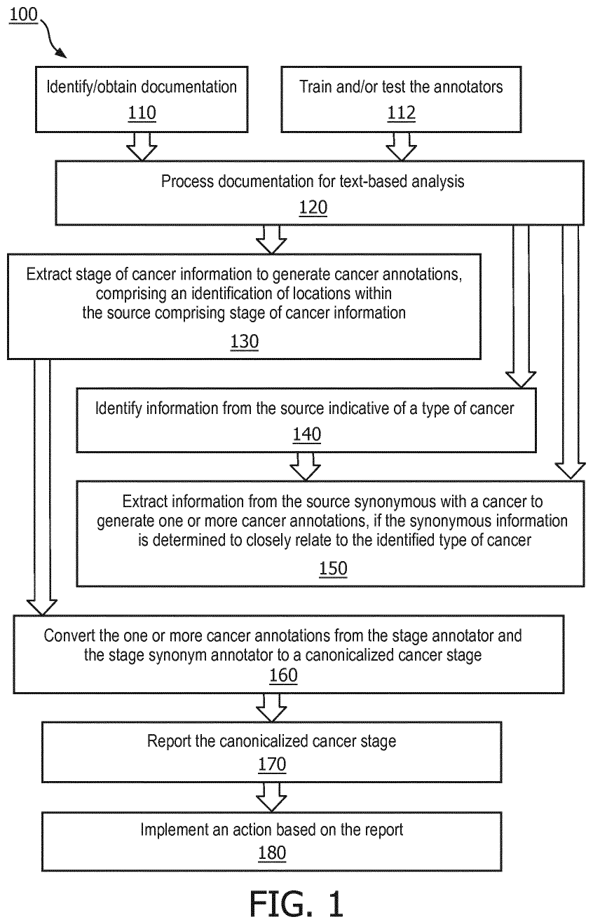

Referring to FIG. 1, in one embodiment, is a flowchart of a method 100 for extracting stage information from text-based documentation and converting the extracted stage information into a standardized format using an annotation system. The methods described in connection with the figures are provided as examples only, and shall be understood not to limit the scope of the disclosure. The annotation system can be any of the systems described or otherwise envisioned herein.

At step 110 of the method, one or more text-based sources are obtained or received by the annotation system. These text-based sources can be any text, document, or other record or source comprising text. According to a preferred embodiment, the text-based sources are digital or digitized sources. For example, the text-based sources may be medical trial information, including the qualifications, parameters, or other information about the trial. As another example, the text-based sources may be clinical records, lab reports, or other medical information about a patient. These are just examples and not meant to be exhaustive. The text-based sources can be provided to the annotation system by an individual or another system. Additionally and/or alternatively, the text-based sources can be retrieved by the annotation system. For example, the annotation system may continuously or periodically access a database, website, or any other resource comprising or providing text-based sources. In the case of trial documents, for example, the documents may be retrieved from a database of medical trials and associated information.

The received or obtained text-based sources may be stored in a local or remote database for use by the annotation system. For example, the annotation system may comprise a database to store the text-based sources, and/or may be in communication with a database storing the text-based sources. These databases may be located with the annotation system or may be located remote from the annotation system, such as in cloud storage and/or other remote storage.

At step 120 of the method, the annotation system processes the text-based sources to prepare them for text-based analysis. The annotation system may process each text-based source as it is received, or may process text-based sources in batches, or may process a text-based source just before it is analyzed in a subsequent step of the method. The text-based sources may be processed using any method or system for processing that facilitates downstream text-based analysis. This processing may include, for example, identification and/or extraction of text from the source, especially if the source comprises content other than text such as images, tables, or other non-text content. The processing may also include normalization of the extracted text, translation of extracted text, and many other forms or kinds of processing. The processed text-based sources, or the processed content therein, may be stored in local or remote storage for subsequent steps of the process.

At step 130 of the method, a stage annotator of the annotation system extracts information from the within the text-based source, or within text extracted from the text-based source, relative to a stage of the patient's cancer to generate one or more cancer annotations. This information comprises, for example, an identification of one or more locations within the text-based source comprising information indicative of a stage of cancer.

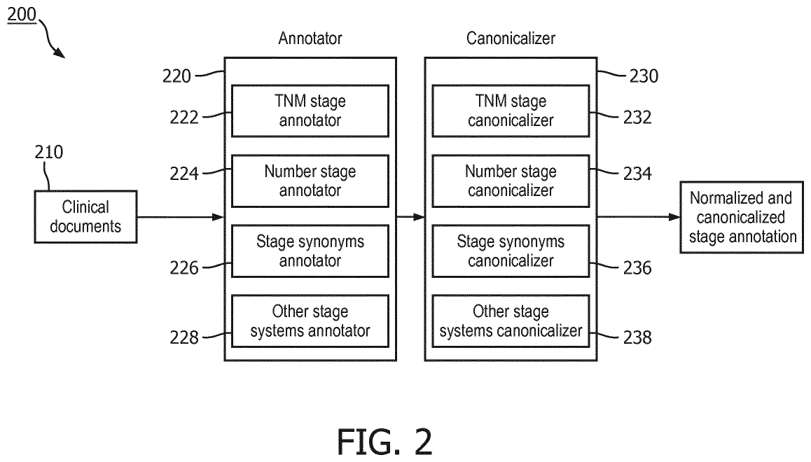

The stage annotator may comprise one or more annotators configured to identify and/or extract cancer stage information from the within the text-based source. Referring to FIG. 2, in one embodiment, is an annotation system 200 which includes an Annotator 220 configured to generate one or more cancer annotations. The annotator 220 receives one or more text-based sources 210 and processes the information to generate one or more cancer annotations.

According to an embodiment, the stage annotator 220 comprises a TNM annotator 222 configured to identify one or more locations within the text-based source comprising information indicative of a TNM classification of a tumor. TNM classification characterizes the anatomical extent of tumors. The “T” of the classification describes the size of the primary tumor and whether it has invaded nearby tissue; the “N” of the classification describes any nearby lymph nodes that may be involved; and the “M” of the classification describes any metastasis of the cancer. A TNM stage is typically written as <prefix>T<grade>N<grade>M<grade> where <prefix> designates whether it is a clinical or pathological stage (or any of a few more variants), and where the three <grade>s describe the primary tumor, lymph nodes, and metastasis. A <grade> is a number between 0 and (up to) 4, followed by an optional letter. The <prefix> and any of the <grade>s are optional, and can be omitted when reviewing a text-based source.

Accordingly, the TNM annotator 222 is configured to recognize all possible combinations of <prefix> and <grades>, taking into account the optionality of each component. The annotator is also configured to recognize enumerations and ranges of stages, such as T1,2 and T2a-c. Note that the actual allowed values for <grade> are defined per cancer type. As described below, there is a relationship between a TNM stage and number staging systems.

According to an embodiment, the stage annotator comprises a number annotator 224 configured to identify one or more locations within the text-based source comprising information indicative of numerical classification of a tumor. Numerical stages are written or provided in text in many different ways and formats. For example, stages may be written as ‘stage I’, ‘stage: I’, ‘stages I and II’, ‘stage I and stage II’, ‘stage Ia to IIIb’, and so on.

According to an embodiment, the number annotator 224 is configured to first detect a single stage without range, such as ‘stage: III’, ‘stage 3’, and so on. The number annotator may be configured or trained by performing a landscape identification of all variants of stage formats identified in text-based sources such as clinical trial document. The number annotator may thus identify a single stage by performing pattern recognition or any other method for identifying text or characters within a text-based source.

After identifying a stage, the number annotator 224 optionally normalizes the identified stage by converting all identified stages to a single standardized format. As one option, the identified stages are all converted to Roman numerals. Thus, stages such as “3” or “three” will be converted to Roman numeral “III.”

The number annotator 224 may thus be further configured to detect a stage range such as stage IIa to IIIb’, ‘stages I and II’, ‘stage: I, II, III’, and so on. The number annotator can be configured to convert the detected stage range to a standardized format. As one option, the identified stage ranges are all converted to Roman numeral ranges. Thus, a stage indicator such as “stages 1 and 2” is converted to “stage I and II.”

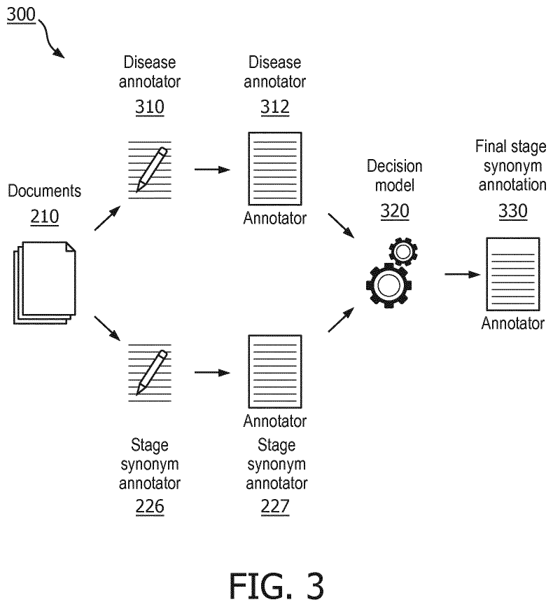

According to an embodiment, the stage annotator comprises a stage synonym annotator 226 configured to identify one or more locations within the text-based source, and/or extract information from one or more locations within the text-based source, comprising information synonymous with a cancer to generate one or more stage synonym annotations. Referring to FIG. 3, in one embodiment, is a flowchart of a process 300 for deriving a state synonym annotation 330 using the stage synonym annotator 226. The stage synonym annotator 226 receives and analyzes information from one or more text-based sources 210.

At step 140 of the method from FIG. 1, a disease annotator 310 of the annotation system identifies and/or extracts information from the within the text-based source indicative of a type of cancer. The disease annotator 310 may be programmed or trained to recognize terminology, phrases, or other information that indicates a type of cancer. For example, the disease annotator 310 may be programmed or trained to identify and/or extract location information such as “neck” or “throat” or “pancreas,” alone or in combination with other terminology, to determine a location or type of cancer. This produces a disease annotation 312 comprising an identification or other characterization of a cancer type.

At step 150 of the method from FIG. 1, a stage synonym annotator 226 identifies one or more locations within the text-based source, and/or extracts information from one or more locations within the text-based source, comprising information synonymous with a cancer to generate one or more stage synonym annotations 227.

Cancer documentation can comprise a wide variety of terminology that describes or otherwise relates to the cancer and is indicative or directly descriptive of a cancer stage. For example, cancer stage synonyms such as ‘locally advanced breast cancer’, or ‘metastatic lung cancer’, and others are also commonly used. These synonyms can be converted to numeral stages. According to an embodiment, these synonyms can be collected and included from multiple cancer stage related documents, such as journals, medical records, and papers. Detecting possible synonym phrases such as ‘metastatic’ alone may not be sufficient, as these phrases are sometimes not describing cancer stage. As an example, phrases such as ‘in situ lung cancer’ means an early stage of lung cancer, but ‘in situ’ alone can mean ‘in place’ which is irrelevant with regard to cancer stage.

Referring to TABLE 1, in one embodiment, are examples of stage synonyms and a numerical stage with which the stage synonym is correlated.

TABLE 1
Examples of Stage Synonyms

| | Number | |
|---|---|---|
| Stage Synonym | Stage | Explanation |
| carcinoma in situ, | 0 | There is a group of abnormal cells in |
| in-situ, CIS | | an area of the body. The cells may |
| | | develop into cancer at some time in |
| | | the future. |
| localized | 1 | Cancer is limited to the place where it |
| | | started, with no sign that it has spread. |
| early stage, | 1, 2 | |
| early-stage | | |
| regional | 3, some | Cancer has spread to nearby lymph |
| | cancer | nodes, tissues, or organs. |
| | type's 2 | |
| locally advanced | 2, 3 | Cancer has spread to nearby lymph |
| | | nodes, tissues, or organs. |
| advanced, secondary, | 4 | Cancer has spread to distant parts of the |
| metastatic, distant | | body. |

The annotator system can be configured to determine whether the stage synonym annotation is sufficiently related to the identified information indicative of a type of cancer. For example, the stage synonym annotator 226 can compare the disease annotation 312 and the stage synonym annotation 227 to determine whether they are compatible. If the stage synonym annotation 227 is compatible with the disease annotation 312, meaning that, for example, the stage synonym is a synonym that is associated with the identified type of cancer, a final stage synonym annotation 330 is generated. For example, a decision model 320 may be utilized to determine whether the stage synonym annotation identified by stage synonym annotator is accurate. As just one example, the decision model may report a stage synonym annotation as being accurate if a cancer label appears very closely (e.g., no more than 2 terms distant) with the detected stage synonym. By combining the two annotations together with the decision model, the stage synonym annotator exhibits good performance The final stage synonym annotation 330 is a cancer annotation that can be utilized in a subsequent step of the process by the annotator system.

According to an embodiment, the stage annotator optionally comprises one or more specialized annotators 228 configured to extract information from the within a text-based source, or within text extracted from a text-based source, relative to a stage of the patient's cancer to generate one or more cancer annotations. The one or more specialized annotators 228 are configured to recognize a specialized cancer stage classification. For example, the specialized annotator 228 may be configured to recognize Ann Arbor staging, Spigelman staging, and/or any other specialized types of cancer stage classification.

The extracted one or more cancer annotations generated by any of the annotators in the annotation system may be stored in a local or remote database for use by the annotation system. For example, the annotation system may comprise a database to store the one or more cancer annotations, and/or may be in communication with a database storing the one or more cancer annotations. These databases may be located with the annotation system or may be located remote from the annotation system, such as in cloud storage and/or other remote storage.

Referring again to FIG. 1, in one embodiment, at step 160 of the method a stage canonicalizer converts the one or more cancer annotations from the annotator to a canonicalized cancer stage. For example, as shown in FIG. 2, the canonicalizer 230 receives the one or more cancer annotations from the annotator 220 and modifies the cancer annotations to a standardized format. The standardized format may be selected or otherwise determined by a user, a system requirement, and/or via other mechanisms. For example, the canonicalizer 230 can be configured or programmed to convert all cancer annotations from the annotator to Roman numerals.

According to an embodiment, the canonicalizer 230 can be configured or programmed to canonicalize different formats of same stage to same format. As another example, the canonicalizer 230 can be configured or programmed to convert different stage systems such as stage synonyms to number stage, as shown in TABLE 1. Without this, ‘stage 1 lung cancer’ will not match ‘early stage lung cancer’, which would omit an important cancer annotation.

Referring to TABLE 2, in one example, is a set of canonicalizers or canonicalization protocols for the canonicalizer 230, which convert different stage indicators to a standardized format. According to an embodiment, the output of two or more canonicalizers or canonicalization protocols can be combined, or two or more canonicalizers or canonicalization protocols can be organized in series, such that the final output of the canonicalizer 230 is a standardized stage extracted from a text-based source. This final output can also comprise a location within the text-based source where an annotation, upon which the standardized stage is based, was identified.

TABLE 2
Examples of canonicalizers

| Stage | | |
|---|---|---|
| System | Canonicalizer | Example |
| TNM stage | range to list of stages | T1-T3 −> T1, T2, T3 |
| number stage | 1. number to roman numerals | stage 3a −> IIIa |
| | 2. expand stage range to list | stages I-III −> I, II, III |
| | of stages | |
| stage | to number stage | See TABLE 1 |
| synonym | | |
| other stages | range to list of stages | Ann arbor stage 1 to 3 −> |
| | | Ann arbor stage 1, 2, 3 |

The standardized stage and/or annotation locations can be stored in a local or remote database for use by the annotation system. For example, the annotation system may comprise a database to store the standardized stage, and/or may be in communication with a database storing the standardized stage. These databases may be located with the annotation system or may be located remote from the annotation system, such as in cloud storage and/or other remote storage.

Referring again to FIG. 1, in one embodiment, at step 170 of the method, the annotation system generates and/or provides a report of the canonicalized cancer stage as generated by the canonicalizer 230. According to an embodiment, the report can further comprise the one or more cancer annotations extracted from the text-based source, and/or the location of the one or more cancer annotations within the text-based source.

The report may be provided via a user interface of the system, which can be any device or system that allows information to be conveyed and/or received, and may include a display, a mouse, and/or a keyboard for receiving user commands. The report may be a visual display, a printed text, an email, an audible report, a transmission, and/or any other method of conveying this information. The report may be provided locally or remotely, and thus the system or user interface may comprise or otherwise be connected to a communications system. For example, the system may communicate a report over a communications system such as the internet or other network.

At optional step 180 of the method, the information contained with the report is utilized to implement one or more subsequent actions. As just one example, the report may be received and reviewed by a healthcare professional. The cancer stage information from the text-based sources provided with regard to a patient, for example, may be utilized by the healthcare professional to determine, confirm, or otherwise inform a treatment for the patient.

As another example, the report may be utilized to extract cancer stage requirements from clinical trial documentation. Since cancer stage requirements are typically provided as free-text in clinical trial documents, a standardized scheme for identifying and reporting cancer stage requirements can be highly beneficial for busy healthcare professional or other clinicians. As an example, the extracted standardized cancer stage information can be stored in a database or otherwise utilized to create a listing of clinical trials. This listing can be utilized, for example, by healthcare professional or other clinicians to identify possible clinical trials for a patient.

The annotation system can be trained using a variety of training methods. For example, a large number of documents (such as clinical trial documents) can be manually annotated as ground truth. The annotator system can then annotate the same set of documents. The system may then compare the manual annotations versus the annotation system annotations, which will show true positives (TPs), false positives (FPs) and false negatives (FNs). The system or an individual can then manually review any false annotations. If an error in the annotator system annotation is detected upon review, the information can be provided back into the annotation system to improve annotation. This process can be repeated until the precision and recall achieves a sufficient level.

For example, method 100 may comprise a training and/or testing step 112. A human reviewer generates, by reviewing the text-based source, a standardized cancer stage. The system compares the reviewer's standardized cancer stage to a standardized cancer stage generated by the annotation system. The system identifies, based on the comparison, any differences between the reviewer's standardized cancer stage and the standardized cancer stage generated by the annotation system. According to an embodiment, if the reviewer's standardized cancer stage and the standardized cancer stage generated by the annotation system do not match, or are not sufficiently similar, a user or a training element of the system can modify one or more of the disease annotator, the stage annotator, the stage synonym annotator, and/or the stage canonicalizer to properly standardize the cancer stage in future iterations.

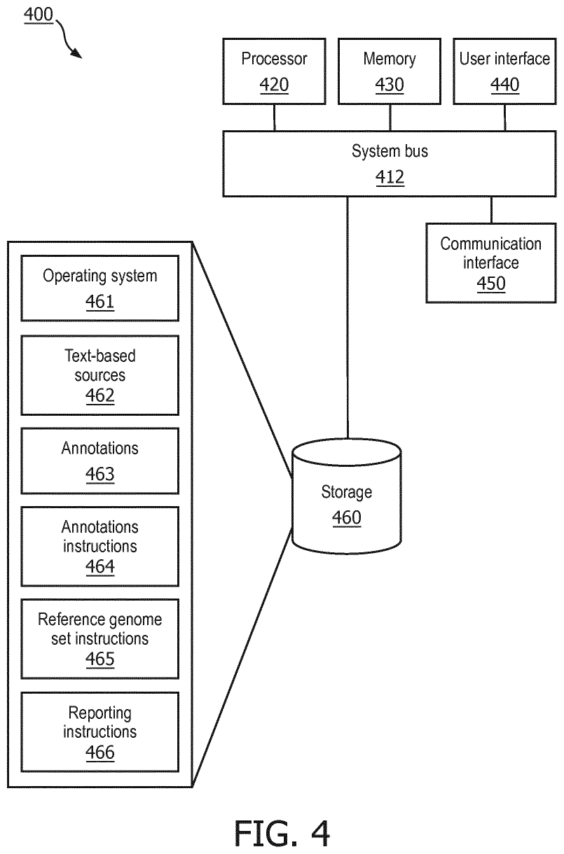

Referring to FIG. 4, in one embodiment, is a schematic representation of an annotation system 400 for generating a genome reference. System 400 may be any of the systems described or otherwise envisioned herein, and may comprise any of the components described or otherwise envisioned herein.

According to an embodiment, system 400 comprises one or more of a processor 420, memory 430, user interface 440, communications interface 450, and storage 460, interconnected via one or more system buses 412. It will be understood that FIG. 4 constitutes, in some respects, an abstraction and that the actual organization of the components of the system 400 may be different and more complex than illustrated.

According to an embodiment, system 400 comprises a processor 420 capable of executing instructions stored in memory 430 or storage 460 or otherwise processing data to, for example, perform one or more steps of the method. Processor 420 may be formed of one or multiple modules. Processor 420 may take any suitable form, including but not limited to a microprocessor, microcontroller, multiple microcontrollers, circuitry, field programmable gate array (FPGA), application-specific integrated circuit (ASIC), a single processor, or plural processors.

Memory 430 can take any suitable form, including a non-volatile memory and/or RAM. The memory 430 may include various memories such as, for example L1, L2, or L3 cache or system memory. As such, the memory 430 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 400. It will be apparent that, in embodiments where the processor implements one or more of the functions described herein in hardware, the software described as corresponding to such functionality in other embodiments may be omitted.

User interface 440 may include one or more devices for enabling communication with a user. The user interface can be any device or system that allows information to be conveyed and/or received, and may include a display, a mouse, and/or a keyboard for receiving user commands In some embodiments, user interface 440 may include a command line interface or graphical user interface that may be presented to a remote terminal via communication interface 450. The user interface may be located with one or more other components of the system, or may located remote from the system and in communication via a wired and/or wireless communications network.

Communication interface 450 may include one or more devices for enabling communication with other hardware devices. For example, communication interface 450 may include a network interface card (NIC) configured to communicate according to the Ethernet protocol. Additionally, communication interface 450 may implement a TCP/IP stack for communication according to the TCP/IP protocols. Various alternative or additional hardware or configurations for communication interface 450 will be apparent.

Storage 460 may include one or more machine-readable storage media such as read-only memory (ROM), random-access memory (RAM), magnetic disk storage media, optical storage media, flash-memory devices, or similar storage media. In various embodiments, storage 460 may store instructions for execution by processor 420 or data upon which processor 420 may operate. For example, storage 460 may store an operating system 461 for controlling various operations of system 400. Storage 460 may also store one or more text-based sources 462 and/or one or more annotations 463.

It will be apparent that various information described as stored in storage 460 may be additionally or alternatively stored in memory 430. In this respect, memory 430 may also be considered to constitute a storage device and storage 460 may be considered a memory. Various other arrangements will be apparent. Further, memory 430 and storage 460 may both be considered to be non-transitory machine-readable media. As used herein, the term non-transitory will be understood to exclude transitory signals but to include all forms of storage, including both volatile and non-volatile memories.

While annotation system 400 is shown as including one of each described component, the various components may be duplicated in various embodiments. For example, processor 420 may include multiple microprocessors that are configured to independently execute the methods described herein or are configured to perform steps or subroutines of the methods described herein such that the multiple processors cooperate to achieve the functionality described herein. Further, where one or more components of system 400 is implemented in a cloud computing system, the various hardware components may belong to separate physical systems. For example, processor 420 may include a first processor in a first server and a second processor in a second server. Many other variations and configurations are possible.

According to an embodiment, storage 460 of annotation system 400 may store one or more algorithms and/or instructions to carry out one or more functions or steps of the methods described or otherwise envisioned herein. For example, processor 420 may comprise, among other instructions, annotation instructions 464, canonicalization instructions 465, and reporting instructions 466.

According to an embodiment, annotation instructions 464 direct the system to generate one or more annotations from one or more text-based sources, which may include an identification of one or more locations within the text-based source that comprises information indicative of a stage of cancer. For example, according to an embodiment, the annotation system receives one or more text-based sources and processes the information to generate one or more cancer annotations. The annotation instructions 464 may include instructions for disease identification, TNM annotation, number annotation, stage synonym annotation, and/or specialized forms of identification or annotation as described or otherwise envisioned herein.

With regard to stage synonym annotation, according to an embodiment, the annotation instructions 464 direct the system to determine whether a stage synonym annotation is sufficiently related to an identified type of cancer, and if so, to generate final stage synonym annotation. For example, the annotation instructions may comprise a comparison or decision model that is utilized to determine whether the stage synonym annotation identified by stage synonym annotator is accurate based on the comparison.

The instructions may direct the system to store the one or more annotations in a local or remote database for retrieval and use by the annotation system. The database may be located with the annotation system or may be located remote from the annotation system, such as in cloud storage and/or other remote storage.

According to an embodiment, canonicalization instructions 465 direct the system to generate canonicalized stage information. For example, according to an embodiment, the canonicalization instructions direct the system to convert the one or more cancer annotations from a non-standardized format to a standardized cancer stage output. The standardized format may be selected or otherwise determined by a user, a system requirement, and/or via other mechanisms. For example, the canonicalization instructions can be configured or programmed to convert all cancer annotations from the annotator to Roman numerals, although many other formats are possible. The canonicalization instructions can also be configured or programmed to generate canonicalized stage information that comprises a location within the text-based source where each annotation, upon which the standardized stage is based, was identified.

The instructions may direct the system to store the canonicalized stage information in a local or remote database for retrieval and use by the annotation system. The database may be located with the annotation system or may be located remote from the annotation system, such as in cloud storage and/or other remote storage.

According to an embodiment, reporting instructions 466 direct the system to generate and/or provide a report of the canonicalized stage information. According to an embodiment, the report can further comprise the one or more cancer annotations extracted from the text-based source, and/or the location of each of the one or more cancer annotations within the text-based source. For example, according to an embodiment, the annotation system generates a report and provides the report via a user interface or via a communications network. The report may be a visual display, a printed text, an email, an audible report, a transmission, and/or any other method of conveying this information. The report may be provided locally or remotely, and thus the system or user interface may comprise or otherwise be connected to a communications system. For example, the system may communicate a report over a communications system such as the internet or other network.

According to an embodiment, a healthcare professional may utilize the provided report to implement one or more subsequent actions. For example, the report may be received and reviewed by a healthcare professional. The cancer stage information from the text-based sources provided with regard to a patient, for example, may be utilized by the healthcare professional to determine, confirm, or otherwise inform a treatment for the patient. As another example, the report may be utilized to extract cancer stage requirements from clinical trial documentation. These and other subsequent actions are possible.

The annotation methods and systems described or otherwise envisioned herein provide numerous advantages over existing systems. It is exceedingly time-consuming and labor-intensive to manually extract cancer stage information from clinical documents. However, the ability to capture cancer stage from a clinical trial document is an essential component of an end-to-end automated matching system.

Precision is the fraction of relevant or accurate instances among retrieved instances. Since cancer stage is a critical criterion which must be matched between a patient and a potential clinical trial, precision of cancer stage identification in the clinical trial information is extremely important. The annotation methods and systems described or otherwise envisioned herein improve precision and therefore enable greater accuracy of matching between patients and clinical trials.

The annotation methods and systems described or otherwise envisioned herein also significantly improve recall, where recall is the fraction of relevant instances that have been retrieved over the total amount of relevant instances. Improved recall by a system directly contributes to improving the recall of clinical trial matching. While some annotators may work well with only some stage systems, the annotation methods and systems described or otherwise envisioned herein function well for all stage systems. It comprises major stage systems such as TNM and number stage system, stage synonyms which are also widely used, and minor stage systems that are used less often.

Accordingly, the annotation methods and systems described or otherwise envisioned herein significantly improve patient treatment. For example, a healthcare professional can utilize the annotation methods or system to identify and/or confirm a cancer stage from medical records for the patient, which will directly inform a course of treatment for the patient, including possibly from the standpoint of initial treatment as well as changes or modifications during a course of treatment. As yet another example, a healthcare professional can utilize the annotation methods or system to more accurately identify the stage criteria found within clinical trials, potentially in an automated manner, which facilitates the matching of a patient to one or more possible clinical trials. This can significantly improve the care of the patient, or at least offer more options for treatment.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A computer-implemented method for generating a standardized cancer stage from a text-based source using an annotation system and matching a patient with a clinical trial or treatment, comprising:
receiving a text-based source comprising information about a patient's medical state or condition;
processing, by a processor, the text-based source for text-based analysis;
extracting, by a stage annotator, information from the text-based source relative to a stage of the patient's cancer to generate one or more cancer annotations, comprising an identification of one or more locations within the text-based source comprising information indicative of a stage of cancer;
identifying, by a disease annotator, information from the text-based source indicative of a type of cancer;
extracting, by a stage synonym annotator, information from the text-based source synonymous with a cancer to generate one or more cancer annotations, if the synonymous information is determined, by a decision model, to closely relate to the identified information indicative of a type of cancer;
converting, by a stage canonicalizer, the one or more cancer annotations from the stage annotator and the stage synonym annotator to a standardized cancer stage; and
reporting the standardized cancer stage, the report comprising information for matching a patient with a clinical trial or treatment including: the standardized cancer stage, the one or more cancer annotations extracted from the text-based source, and/or the location of each of the one or more cancer annotations within the text-based source.

2. The method of claim 1, further comprising implementing an action based on the report.

3. The method of claim 2, wherein the action is implementation of a treatment plan by a healthcare professional.

4. The method of claim 2, wherein the action is identification of a suitable clinical trial for the patient based on the cancer stage extracted from the clinical trial.

5. The method of claim 1, wherein the stage annotator comprises: (i) a TNM annotator configured to identify one or more locations within the text-based source comprising information indicative of a TNM classification of a tumor;
and (ii) a number annotator configured to identify one or more locations within the text-based source comprising information indicative of numerical classification of a tumor.

6. The method of claim 1, wherein the standardized cancer stage comprises a Roman numeral.

7. The method of claim 1, further comprising the step of testing the annotation system by: (i) generating, by a reviewer reviewing the text-based source, a standardized cancer stage; (ii) comparing the reviewer's standardized cancer stage to the standardized cancer stage generated by the annotation system; (iii) identifying, from the comparison, any differences between the reviewer's standardized cancer stage and the standardized cancer stage generated by the annotation system; and (iv) modifying one or more of the disease annotator, the stage annotator, the stage synonym annotator, and/or the stage canonicalizer if the reviewer's standardized cancer stage and the standardized cancer stage generated by the annotation system do not match.

8. The method of claim 1, wherein the information from the text-based source synonymous with a cancer comprises information describing a physical state of a tumor.

9. A system configured to generate a standardized cancer stage from a text-based source for matching a patient with a clinical trial or treatment, comprising:
a plurality of text-based sources;
a processor configured to: (i) extract information from the text-based source relative to a stage of the patient's cancer to generate one or more cancer annotations, comprising an identification of one or more locations within the text-based source comprising information indicative of a stage of cancer; (ii) identify information from the text-based source indicative of a type of cancer; (iii) extract information from the text-based source synonymous with a cancer to generate one or more cancer annotations, if the synonymous information is determined to closely relate to the identified information indicative of a type of cancer; (iv) convert the one or more cancer annotations from the stage annotator and the stage synonym annotator to a standardized cancer stage; and (v) generate a report of the standardized cancer stage, comprising information for matching a patient with a clinical trial or treatment including: the standardized cancer stage, the one or more cancer annotations extracted from the text-based source, and/or the location of the one or more cancer annotations within the text-based source; and
a user interface configured to communicate the report of the standardized cancer stage to a user.

10. The system of claim 9, wherein the processor is configured to: (i) identify one or more locations within the text-based source comprising information indicative of a TNM classification of a tumor; and/or (ii) identify one or more locations within the text-based source comprising information indicative of numerical classification of a tumor.

11. The system of claim 9, wherein the standardized cancer stage comprises a Roman numeral.

12. The system of claim 9, wherein the processor is configured to: (i) compare the standardized cancer stage to a standardized cancer stage generated by a human reviewer; (ii) identify any differences between the standardized cancer stage and the standardized cancer stage generated by the human reviewer; and (iii) modify the system if the standardized cancer stage and the standardized cancer stage generated by the human reviewer are not a match.

13. The system of claim 9, wherein the information from the text-based source synonymous with a cancer comprises information describing a physical state of a tumor.

14. The system of claim 9, wherein the plurality of text-based sources comprises clinical documents about one or more patients.

15. The system of claim 9, wherein the plurality of text-based sources comprises documentation about one or more clinical trials.
